(19) United States
(12) Patent Application Publication
(10) Pub. No.: US 2026/0345351 A1
(43) Pub. Date: Nov. 5, 2026

(54) INNOVATION IN ROBOTIC HAND PROSTHESIS

(71) Applicant: OZYEGIN UNIVERSITESI, Istanbul (TR)

(72) Inventors: Ramazan UNAL, Istanbul (TR); Mahsa SARRAFIKHOSROWSHAH, Istanbul (TR)

(73) Assignee: OZYEGIN UNIVERSITESI, Istanbul (TR)

(21) Appl. No.: 19/101,899

(22) PCT Filed: Oct. 17, 2022
(86) PCT No.: PCT/TR2022/051147

§ 371 Date: Feb. 7, 2025

(30) Foreign Application Priority Data
Aug. 8, 2022 (TR) 2022/012480

(51) Int. Cl. A61F 2/58; A61F 2/50; A61F 2/70

(52) U.S. Cl. CPC A61F 2/586; A61F 2/70; A61F 2002/5093; A61F 2002/704

(57) ABSTRACT

A robotic hand prosthesis for allowing amputees who have lost their entire hand to perform their activities of daily living, comprising at least one lower body, a thumb connected to the lower body, an index finger and at least one motion group, at least one motor B assisting the motion of the thumb, at least one motor A assisting the motion of the index finger and a motor C assisting the motion group to the desired position, wherein the robotic hand is configured to perform the hand motion by the shoulder of the amputee after the main tasks during the use of the robotic hand prosthesis are provided with preconditioning.

### CROSS REFERENCE TO RELATED APPLICATIONS

This application is a national stage entry of International Application No. PCT/TR2022/051147, filed on Oct. 17, 2022, which is based upon and claims foreign priority to Turkey Patent Application No. 2022/012480, filed on Aug. 8, 2022, the entire contents of which are incorporated herein by reference.

### TECHNICAL FIELD

The invention relates to innovation in a bio-inspired robotic hand prosthesis powered by body power for allowing amputees who have lost their entire hand to perform their activities of daily living.

### BACKGROUND

Today, an amputee person who has lost their entire hand can carry out their activities of daily living with the help of prosthetic robotic hands. Prosthetic robot hands allow a person who has lost their hand to gain most of the functionality and dexterity they had before the loss of the limb. The human hand performs the functions of grasping, squeezing, relaxing, feeling, etc. at an excellent level, and the design of prosthetic robot hands is a very good example of the real thing with these features. Since the human hand is the inspiration for robot hands, which is an electro-mechanical system formed by the combination of many moving joints, these mechanisms are described as bio-inspired. In robotic hand prostheses, the motion is driven by a mechanical structure, and the control of the system is usually provided by electrical parts.

There is a document with application number US2015112448A1 in the state of the art. In this document, the invention is found in the technical field of prosthetic devices. More particularly, the prosthesis comprises a hybrid prosthesis capable of controlling the motion set of a prosthetic device via EMG signals as well as the motion of the respective shoulder and elbow of the prosthetic hand user. The mechanical functions of the present invention are controlled by either a shrugging motion, an elbow lift or a collapsing motion, or both. The forces provided by these motions are transferred to the hand using a Bowden cable design. The operator can then choose what this motion will do in the hand function, allowing the system to adapt to the personal capability and desired task of the amputee person.

There is a document in the literature with application number US2021386562A1. The invention relates to a hybrid-driven prosthetic device. An upper extremity prosthetic device comprises a hybrid-driven prosthetic device comprising one or more actuators fixed to a first arm portion and one or more wires extending from one or more actuators along one or more cable paths defined along the first arm portion. The alternative or additional upper limb prosthetic device has a terminal device/device assembly wherein one or more cables are configured to be actuated by one or more actuators and further configured to be actuated by rotating the second arm portion toward the first arm portion. In the said invention, the motion is made by moving the elbow joint and by receiving power from the body.

All the problems mentioned above have made it necessary to make an innovation in the relevant technical field as a result.

### SUMMARY

The present invention relates to innovation in robotic hand prostheses to eliminate the abovementioned disadvantages and to bring new advantages to the related technical field.

An object of the invention is to provide a robotic hand prosthesis that enables the actuators to only to bring the fingers to the desired position and meets the main forces through the shoulder or elbow of the amputee person, thus supporting the acceptance of the prosthesis by the user and the self-confidence of the individual by reducing both the mental and physical effort of the user.

Another object of the invention is to provide a robotic hand prosthesis that is easy to use, lightweight, and allows energy efficiency since heavy and powerful motors are not needed during use.

In order to achieve all the objects mentioned above and that will emerge from the following detailed description, the present invention is a robotic hand prosthesis for allowing amputees who have lost their entire hand to perform their activities of daily living, comprising at least one lower body, a thumb connected to the said lower body, an index finger and at least one motion group, at least one motor B assisting the motion of the said thumb, at least one motor A assisting the motion of the said index finger and a motor C assisting the motion group to the desired position. Accordingly, its novelty is that it is configured to perform the hand motion by the shoulder of the amputee after the main tasks during the use of the said robotic hand prosthesis are provided with preconditioning. Thus, it is aimed to provide a robotic hand prosthesis that supports the acceptance of the prosthesis by the user and the self-confidence of the individual by reducing both the mental and physical effort of the user by ensuring that the force is met over the shoulder of the non-prosthetic side and thus by enabling the amputee to be active.

A possible embodiment of the invention is characterized in that the said preconditioning process is provided via a mobile device. Thus, it is to present a hybrid, easy-to-control and lightweight robotic hand prosthesis that provides the continuation of the preconditioned motion of movements and tasks to be covered by the amputee person's shoulder.

Another possible embodiment of the invention is characterized in that it comprises a spring A provided on the lower body and connected to the robotic hand prosthesis tendons and the index finger. Thus, it is to provide a robotic hand prosthesis that allows the index finger to return to its old/default position and/or anatomical position.

Another possible embodiment of the invention is characterized in that it comprises a spring B provided on the lower body and connected to the robotic hand prosthesis tendons and the thumb. Thus, it is to provide a robotic hand prosthesis that allows the thumb to return to its old/default position and/or anatomical position.

Another possible embodiment of the invention is characterized in that it comprises a spring C provided on the lower body and connected to the robotic hand prosthesis tendons and the motion group. Thus, it is to present a robotic hand prosthesis that allows the motion group consisting of the middle, ring, and little fingers to return to their old/default position and/or anatomical positions.

Another possible embodiment of the invention is characterized in that it comprises an upper body provided on the robotic hand prosthesis and to cover the robotic hand prosthesis. Thus, it is to provide a robotic hand prosthesis that allows the robotic hand prosthesis to be covered and to look stylish so that it is not damaged.

Another possible embodiment of the invention is characterized in that it comprises a processor assisting in the operation of the robotic hand prosthesis. Thus, it is particularly important to provide a robotic hand prosthesis that supports the control and preconditioning of the actuators.

### BRIEF DESCRIPTION OF THE DRAWINGS

FIGURE shows a representative overview of the innovation in the robotic hand prosthesis of the invention.

### REFERENCE NUMBERS GIVEN IN THE FIGURE

100 Robotic Hand Prosthesis
10 Upper Body
20 Lower Body
21 Connection Part
30 Index Finger
31 First Articulation of Index Finger
32 Second Articulation of Index Finger
33 Third Articulation of Index Finger
34 First Joint of Index Finger
35 Second Joint of Index Finger
36 Third Joint of Index Finger
37 Spring A
38 Motor A
40 Thumb
41 First Articulation of Thumb
42 Third Articulation of Thumb
43 First Joint of Thumb
44 Third Joint of Thumb
45 Spring B
46 Motor B
50 Motion Group
51 First Articulation of Motion Group
52 Second Articulation of Motion Group
53 Third Articulation of Motion Group
54 First Joint of Motion Group
55 Second Joint of Motion Group
56 Third Joint of Motion Group
57 Spring C
58 Motor C

### DETAILED DESCRIPTION OF THE EMBODIMENTS

In this detailed description, the innovation in the robotic hand prosthesis of the invention is explained with examples that do not have any limiting effect only for a better understanding of the subject.

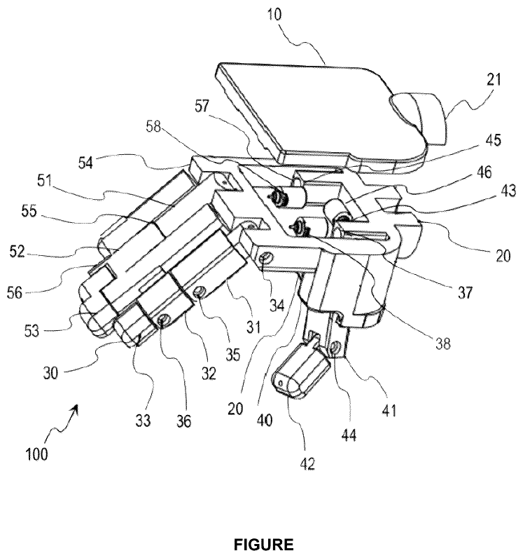

FIG. 1 shows the robotic hand prosthesis (100) of the invention. For allowing amputees to perform their activities of daily living, the said robotic hand prosthesis (100) consists of at least one lower body (20), a thumb (40) connected to the said lower body (20), an index finger (30) and at least one motion group (50) positioned on the side of the said index finger (30) which does not include the said thumb (40). The invention provides at least one motor B (46) for assisting the motion of the thumb (40), at least one motor A (38) for assisting the motion of the said index finger (30), and at least one motor C (58) for assisting in bringing the motion group (50) to the desired position. It is ensured that the upper body (10) on the robotic hand prosthesis (100) and the elements provided especially on the lower body (20) are covered to prevent damage. It is also aimed to obtain a stylish image with the upper body (10) and ensure the integrity of the body.

The said robotic hand prosthesis (100) is connected to the limb of the person with the connection part (21) and the main tasks and finger motion forces during its use are ensured to be performed by the shoulder of the amputee person.

The thumb (40) in the robotic hand prosthesis (100) consists of a structure with two degrees of freedom with rotational and translational motion, the first articulation of the thumb (41) and the third articulation of the thumb (42). The first articulation of the thumb (41) helps to perform the grip motion more accurately for anthropomorphic design. The said third articulation of the thumb (42) is necessary for the pinching motion and also makes it possible to provide the catching motion. The said first articulation of the thumb (41) is connected to the lower body by means of the first joint of the thumb (43). The first joint of the thumb (43) is the joint in which it essentially slides on the thumb (40), providing rotational and/or sliding motions. The first articulation of the thumb (41) and the third articulation of the thumb (42) are connected to each other by a joint called the third joint of the thumb (44).

With the robotic hand prosthesis (100), the thumb (40), the index finger (30) and the motion group (50) work in coordination to make the tasks such as grasping, pinching, holding motions or holding an object, changing its position more efficient. The index finger (30) is provided in three parts, namely the first articulation of the index finger (31), the second articulation of the index finger (32), and the third articulation of the index finger (33) and consists of three degrees of freedom embodiment.

The said first articulation of the index finger (31) allows small objects to be held by the pinching motion and it is necessary to capture the motion. The second articulation of the index finger (32) allows to make the motion more accurately and precisely. The third articulation of the index finger (33) is required for gripping motion and more accurate grip again. The first articulation of the index finger (31) is connected to the lower body (20), that is, to the palm, through the first joint of the index finger (34). The second joint of the index finger (35) is the joint that enables the second articulation of the index finger (32) to be connected to the first articulation of the index finger (31). The third articulation of the index finger (33) and the second articulation of the index finger (32) are connected by the joint called the third articulation of the index finger (36).

The robotic hand prosthesis (100) used by the amputee person is provided as a single finger with the middle finger, ring finger, and little finger combined in order to convey all the feeling of a real hand to the user and to allow all the tasks that can be done with a real hand to be carried out in the easiest, lightest and compact way. The said middle finger, ring finger, and little finger are used as a single finger by the motion group (50) and the said motion group (50) is provided on the lower body (20). The motion group (50) includes the first articulation of motion group (51), the second articulation of motion group (52), and the third articulation of motion group (53), providing a three-degrees-of-freedom motion.

The motion group (50), which consists of the combination of the middle finger, the ring finger, and the little finger, enables the grip to be realized more accurately thanks to the three fingers. The first articulation of the motion group (51) is connected to the lower body (20) by the rotational joint of the first joint of the motion group (54). The first articulation of the motion group (51) and the second articulation of the motion group (52) are connected to each other by a rotational joint called the second joint of the motion group (55). The second articulation of the motion group (52) and the third articulation of the motion group (53) are connected to each other by the third joint of the motion group (56). The third joint of the motion group (56) is a rotational joint just like any other joint.

For each task in performing daily activities in the robotic hand prosthesis (100), the thumb (40), index finger (30), and motion group (50) are brought to their predetermined positions by means of 3 motors placed in the palm of the robotic hand prosthesis (100), that is, in the lower body (20). The 3 motors mentioned are motor A (38), motor B (46), and motor C (58), preferably DC (direct current) motors. DC motors are also called servo motors in the literature. The aforementioned motor A (38) is used to perform the preconditioned task and to move the index finger (30) to the desired position. The said motor B (46) helps the thumb (40) and the said C motor (58) to support the motion group (50) to fulfill the desired, preconditioned motions. Compression and gripping forces are provided through tendons attached to the opposite shoulder of the amputee person to accomplish the tasks. Thus, the person using the robotic hand prosthesis (100) is included in the realization of the motions and tasks, and thus, it is ensured that the amputee person easily adapts to the robotic hand prosthesis (100) and regains their self-confidence by reducing their mental and physical effort.

In the robotic hand prosthesis (100), there is a spring A (37) in the index finger (30), a spring B (45) in the thumb (40), and a spring C (57) in the motion group (50). Spring A (37), spring B (45), and spring C (57) are used to return the thumb (40), the index finger (30), and the motion group (50) to their old/default position and/or anatomical positions, respectively. Spring A (37), spring B (45), and spring C (57) follow their respective motors (will be disclosed below) in the embodiment.

The most important point of the invention is that the robotic hand prosthesis (100) is preconditioned for the task desired to be performed, and after being conditioned, the robotic hand performs the conditioned task with the motion given from the person's shoulder. Actuators such as motor A (38), motor B (46) and motor C (58) used in robotic hand prostheses (100) are only used to bring the fingers to the desired position. To explain in more detail, tasks such as holding a cup, and moving items from one box to another are transferred to the robotic hand prosthesis (100) by preconditioning. However, actions such as carrying out the holding process or transferring items from one box to another are performed by moving the shoulder of the amputee person.

The preconditioning and commands required for motion can be sent via a mobile device such as a mobile phone application. With the preconditioning performed on the mobile device, it is aimed to facilitate the use of robotic hand prostheses (100) and to obtain a prosthesis suitable for the developing technology. A processor can be provided on the robotic hand prosthesis (100) to perform a part of the motion and to use other features of the robotic hand prosthesis (100) with the preconditioning to be made on all this mobile device.

In addition, preconditioning can be met through signals to be received from devices such as audio, EMG, EEG, Bluetooth, and so on. Since the thumb (40) in the robotic hand prosthesis (100) can perform both sliding and rotation motions in the best way, it is more similar to natural thumb motions. This hand structure is printed on a 3-dimensional printer using biocompatible material. In addition, since the shoulder of the amputee person is also used during the operation of the robotic hand prosthesis (100), there is no need for heavy and powerful motors in the structure. For this reason, robotic hand prosthesis (100) is lighter and more compact than those in the current art thanks to its production from a 3-dimensional printer.

The scope of protection of the invention is specified in the attached claims and cannot be limited to those explained for sampling purposes in this detailed description. It is evident that a person skilled in the art may exhibit similar embodiments in the light of the above-mentioned facts without drifting apart from the main theme of the invention.

1. A robotic hand prosthesis for allowing amputees who have lost their entire hand to perform their activities of daily living, comprising at least one lower body, a thumb connected to the lower body, an index finger and at least one motion group, at least one motor B assisting the motion of the thumb, at least one motor A assisting the motion of the index finger and a motor C assisting the motion group to the desired position, wherein the robotic hand is configured to perform a hand motion by the shoulder of an amputee after one or more main tasks during a use of the robotic hand prosthesis are provided with preconditioning.

2. The robotic hand prosthesis according to claim 1, wherein the preconditioning process is provided through a mobile device.

3. The robotic hand prosthesis according to claim 1, wherein the robotic hand comprises a spring A provided on the lower body and connected to the tendons of the robotic hand prosthesis and the index finger.

4. The robotic hand prosthesis according to claim 1, wherein it the robotic hand comprises a spring B provided on the lower body and connected to the tendons of the robotic hand prosthesis and the thumb.

5. The robotic hand prosthesis according to claim 1, wherein the robotic hand comprises a spring C provided on the lower body and connected to the tendons of the robotic hand prosthesis and the motion group.

6. The robotic hand prosthesis according to claim 1, wherein the robitic hand comprises an upper body provided on the robotic hand prosthesis and to cover the robotic hand prosthesis.

7. The robotic hand prosthesis according to claim 1, wherein the operation of the robotic hand prosthesis is supported by a processor.
